(19) United States
(12) Patent Application Publication
(10) Pub. No.: US 2026/0144967 A1
(43) Pub. Date: May 28, 2026

(54) BALLOON CATHETER

(71) Applicant: KANEKA CORPORATION, Osaka (JP)

(72) Inventors: Kazuki Matsufuji, Osaka (JP); Masahiro Kojima, Osaka (JP); Takahisa Hamabuchi, Osaka (JP)

(73) Assignee: KANEKA CORPORATION, Osaka (JP)

(21) Appl. No.: 19/424,379

(22) Filed: Dec. 18, 2025

Related U.S. Application Data
(63) Continuation of application No. PCT/JP2024/022199, filed on Jun. 19, 2024.

(30) Foreign Application Priority Data
Jun. 22, 2023 (JP) 2023-102819

(51) Int. Cl. A61M 25/10; A61M 25/00; A61M 25/09

(52) U.S. Cl. CPC A61M 25/1011; A61M 2025/004; A61M 2025/09008

(57) ABSTRACT

A balloon catheter including a shaft extending in a longitudinal direction from a proximal side toward a distal side and a plurality of balloons disposed at a distal portion of the shaft is provided. The shaft has, in a cross-section at the distal end of the shaft and perpendicular to the longitudinal direction, an inner lumen and a lumen group constituted by a plurality of outer lumens disposed on the outer side with respect to the inner lumen and aligned in a circumferential direction of the inner lumen. The shaft includes, in a cross-section perpendicular to the longitudinal direction, a portion in which a distance between a center of a first outer lumen and a center of a second outer lumen is larger than a distance between the center of the first outer lumen and a center of a third outer lumen.

### TECHNICAL FIELD

One or more embodiments of the present invention relate to a balloon catheter.

### BACKGROUND

A stenosed site hardened by calcification or the like is formed in the inner wall of a blood vessel, thereby causing diseases such as angina pectoris and myocardial infarction. One of treatments for these diseases is angioplasty, in which a balloon catheter is used to dilate a stenosed site. Angioplasty is a minimally invasive treatment that does not require thoracotomy, such as a bypass operation, and is widely performed.

There is a disease called aortic valve stenosis, in which an aortic valve becomes hard due to calcification or the like, the aortic valve becomes difficult to open, and the flow of blood is blocked. As a treatment for aortic valve stenosis, a method in which a bioprosthetic valve (artificial valve) is placed by a surgical open-chest operation and a catheter and a hardened aortic valve is replaced with the bioprosthetic valve is sometimes used.

Implanted bioprosthetic valves deteriorate over time due to calcification, wear, and the like. When an implanted bioprosthetic valve deteriorates, the bioprosthetic valve needs to be replaced. In a technique that has been studied for replacement of bioprosthetic valves, a balloon catheter having a braid or a plurality of balloon catheters is used to apply a high pressure to an indwelling bioprosthetic valve to deform or destroy the valve, thereby dilating the lumen of the valve, and then a new bioprosthetic valve is indwelled inside the deformed or destroyed bioprosthetic valve by transcatheter aortic valve replacement or the like.

As a catheter that is used for dilation of hardened stenosed sites and for indwelling of bioprosthetic valves and that includes a balloon inflatable by a high pressure, for example, PTL 1 discloses a balloon catheter including a plurality of balloon members and in which a plurality of outer balloon members are disposed so as to surround the outer surface of an inner balloon member, and PTL 2 discloses a device that includes a perfusion balloon having an internal passage and a balloon disposed in the internal passage of the perfusion balloon.

### Patent Literature

PTL 1: US Patent Application Publication No. 2012/0209375

PTL 2: Japanese Unexamined Patent Application Publication (Translation of PCT Application) No. 2018-536474

A shaft of a catheter, such as the conventional catheters described in PTL 1 and PTL 2, including a plurality of balloons may be configured to have a plurality of lumens such as inflation lumens connected to respective balloons and through which a fluid to be supplied into the lumens of the balloons passes and a guidewire lumen through which a guidewire is to be inserted. If a shaft has a plurality of lumens, when a balloon catheter is inserted into a lumen in a living body such as a blood vessel, it is difficult to sufficiently transmit a force applied from the hand side (proximal side) of the shaft to a leading end (distal end) of the shaft, and it is difficult to insert the balloon catheter.

### SUMMARY

In view of the above-described circumstances, a balloon catheter in which a force applied from the proximal side of a shaft is easily transmitted to the distal end of the shaft and having favorable force transmissibility is provided.

A balloon catheter according to one or more embodiments of the present invention capable of addressing the above is as follows.
[1] A balloon catheter including:
a shaft extending from a proximal side toward a distal side in a longitudinal direction; and
a plurality of balloons disposed at a distal portion of the shaft,
in which, in a cross-section at a distal end of the shaft and perpendicular to the longitudinal direction, the shaft has an inner lumen and a lumen group constituted by a plurality of outer lumens disposed on an outer side with respect to the inner lumen to be aligned in a circumferential direction of the inner lumen,
in which, in a cross-section at a distal end of the shaft and perpendicular to the longitudinal direction, the lumen group includes a first outer lumen, a second outer lumen disposed adjacent to the first outer lumen in a circumferential direction of the inner lumen, and a third outer lumen disposed adjacent to the first outer lumen and on a side opposite to the second outer lumen in a circumferential direction of the inner lumen, and
wherein, in a cross-section perpendicular to the longitudinal direction, the shaft includes a portion in which a distance between a center of the first outer lumen and a center of the second outer lumen is larger than a distance between a center of the first outer lumen and a center of the third outer lumen.
[2] The balloon catheter according to [1], in which, in a cross-section perpendicular to the longitudinal direction, the shaft includes a portion in which a distance between a center of the first outer lumen and a center of the second outer lumen is larger than a longitudinal diameter of each of the outer lumens constituting the lumen group.
[3] The balloon catheter according to [1] or [2], in which the shaft has a distal region, which is a region on a distal side, and a proximal region, which is a region located on a proximal side with respect to the distal region, and
in which, in a cross-section in the proximal region and perpendicular to the longitudinal direction, a distance between a center of the first outer lumen and a center of the second outer lumen is larger than a distance between a center of the first outer lumen and a center of the third outer lumen.
[4] The balloon catheter according to [3], in which a length of the proximal region is equal to or less than a length of the distal region in the longitudinal direction.
[5] The balloon catheter according to any one of [1] to [4],
in which the shaft has a distal region, which is a region on a distal side, and a proximal region, which is a region located on a proximal side with respect to the distal region, and
in which a distance between a center of the shaft and a center of the inner lumen in a cross-section in the proximal region and perpendicular to the longitudinal direction is larger than a distance between a center of the shaft and a center of the inner lumen in a cross-section in the distal region and perpendicular to the longitudinal direction.
[6] The balloon catheter according to any one of [3] to [5], in which the shaft has, in the proximal region and in a region between the outer lumens adjacent to each other in a circumferential direction of the inner lumen, an opening through which the inner lumen is in communication with a region outside the shaft.
[7] The balloon catheter according to [6], in which the opening is located in a region between the first outer lumen and the second outer lumen in a circumferential direction of the inner lumen.
[8] The balloon catheter according to any one of [3] to [7], in which, in a cross-section in the proximal region and perpendicular to the longitudinal direction, a distance between a center of the shaft and a center of the inner lumen is larger than a shortest distance between an outer edge of the inner lumen and an outer edge of the shaft.
[9] The balloon catheter according to any one of [3] to [8], in which, in a cross-section in the proximal region and perpendicular to the longitudinal direction, a shortest distance between a center of each of the outer lumens constituting the lumen group and a center of the shaft is larger than a center-to-center distance between the outer lumens adjacent to each other in a circumferential direction of the inner lumen.
[10] The balloon catheter according to any one of [3] to [9], in which, in a cross-section in the proximal region and perpendicular to the longitudinal direction, a shortest distance between a center of each of the outer lumens constituting the lumen group and a center of the shaft is larger than a shortest distance between an outer edge of each of the outer lumens and an outer edge of the shaft.
[11] The balloon catheter according to any one of [1] to [10],
in which the inner lumen is a guidewire lumen through which a guidewire is to be inserted, and
in which the outer lumens constituting the lumen group are inflation lumens through which a fluid to be supplied to lumens of the balloons passes, the inflation lumens each being in communication with a corresponding one of lumens of the plurality of balloons.
[12] The balloon catheter according to [11], further including:
a guidewire tube having a lumen in communication with the guidewire lumen,
in which the guidewire tube is disposed in a lumen of one of the balloons.
[13] The balloon catheter according to any one of [1] to [12], in which the balloon catheter is to be used for dilating an aortic valve, deforming a bioprosthetic valve placed in a heart, or destroying the bioprosthetic valve.

### Advantageous Effects of Invention

According to the aforementioned balloon catheter, since the shaft includes a portion in which the distance between the center of the first outer lumen and the center of the second outer lumen is larger than the distance between the center of the first outer lumen and the center of the third outer lumen, the wall thickness of the portion of the shaft between the first outer lumen and the second outer lumen is larger than the wall thickness of the portion of the shaft between the first outer lumen and the third outer lumen. As a result, the rigidity of the portion of the shaft between the first outer lumen and the second outer lumen is increased, and the force applied from the proximal side of the shaft is easily transmitted to the distal end of the shaft. Thus, it is possible to provide a balloon catheter having favorable force transmissibility.

### BRIEF DESCRIPTION OF DRAWINGS

FIG. 1 is a side view of a balloon catheter according to one or more embodiments of the present invention.

FIG. 2 is a side view of balloons of the balloon catheter illustrated in FIG. 1.

FIG. 3 is a side view of the shaft in a distal region of the balloon catheter illustrated in FIG. 1.

FIG. 4 is a cross-sectional view of the shaft illustrated in FIG. 3 taken along the line IV-IV.

FIG. 5 is a side view of a shaft in a proximal region of the balloon catheter illustrated in FIG. 1.

FIG. 6 is a cross-sectional view of the shaft illustrated in FIG. 5 taken along the line VI-VI.

### DETAILED DESCRIPTION

Hereinafter, one or more embodiments of the present invention will be described on the basis of embodiments, but the present invention is not limited to the following embodiments, and it is of course possible to carry out the present invention by adding modifications, as appropriate, within a range that can be adapted to the gist described above and below, and all of them are included in the technical scope of the present invention. In the drawings, hatching, reference signs of members, and the like may be omitted for convenience. In such a case, the description and other drawings are referred to. In addition, the dimensions of various members in the drawings may be different from actual dimensions thereof in order to facilitate understanding of the features of one or more embodiments of the present invention.

A balloon catheter according to one or more embodiments of the present invention includes a shaft extending from a proximal side toward a distal side in a longitudinal direction and a plurality of balloons disposed at a distal portion of the shaft. In a cross-section at a distal end of the shaft and perpendicular to the longitudinal direction, the shaft has an inner lumen and a lumen group constituted by a plurality of outer lumens disposed on the outer side with respect to the inner lumen to be aligned in a circumferential direction of the inner lumen. In a cross-section at the distal end of the shaft and perpendicular to the longitudinal direction, the lumen group includes a first outer lumen, a second outer lumen disposed adjacent to the first outer lumen in the circumferential direction of the inner lumen, and a third outer lumen disposed adjacent to the first outer lumen and on the side opposite to the second outer lumen in the circumferential direction of the inner lumen. In a cross-section perpendicular to the longitudinal direction, the shaft includes a portion in which a distance between a center of the first outer lumen and a center of the second outer lumen is larger than a distance between the center of the first outer lumen and a center of the third outer lumen.

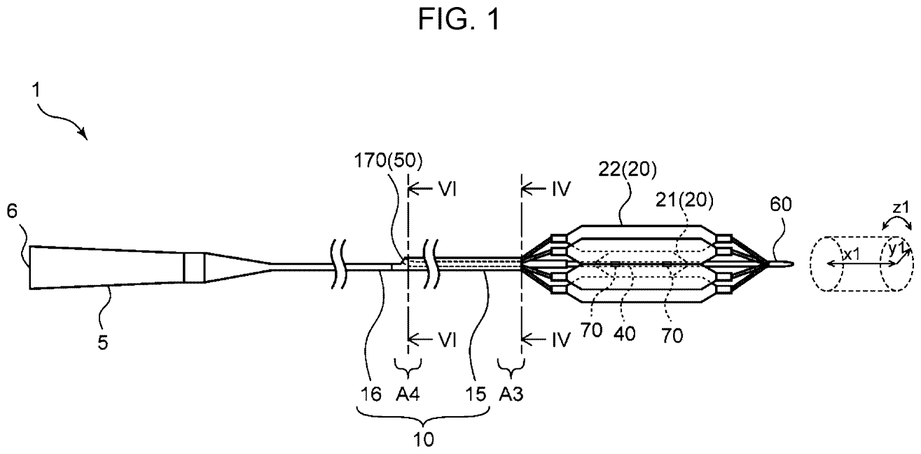

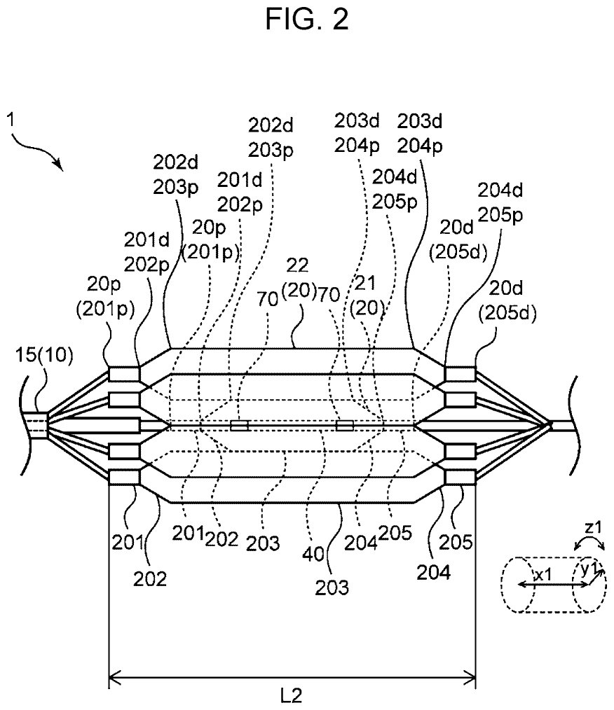

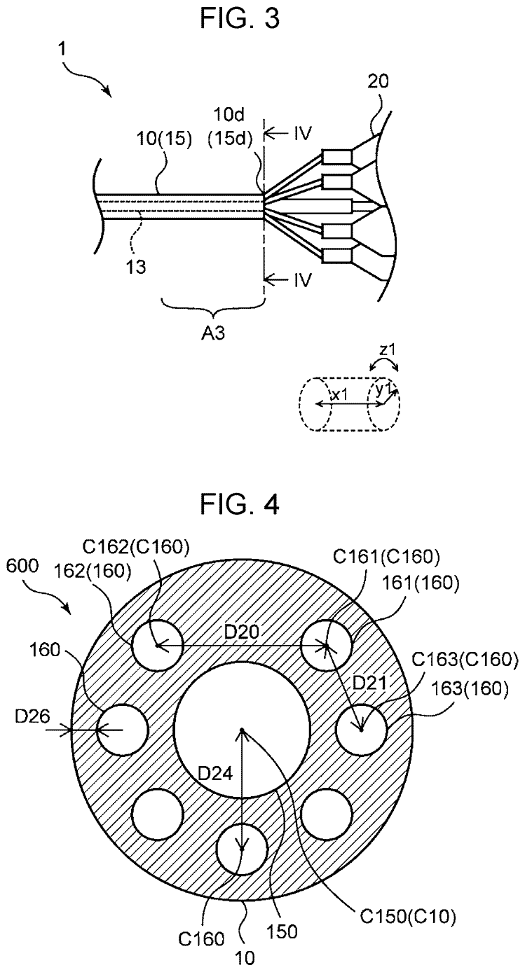

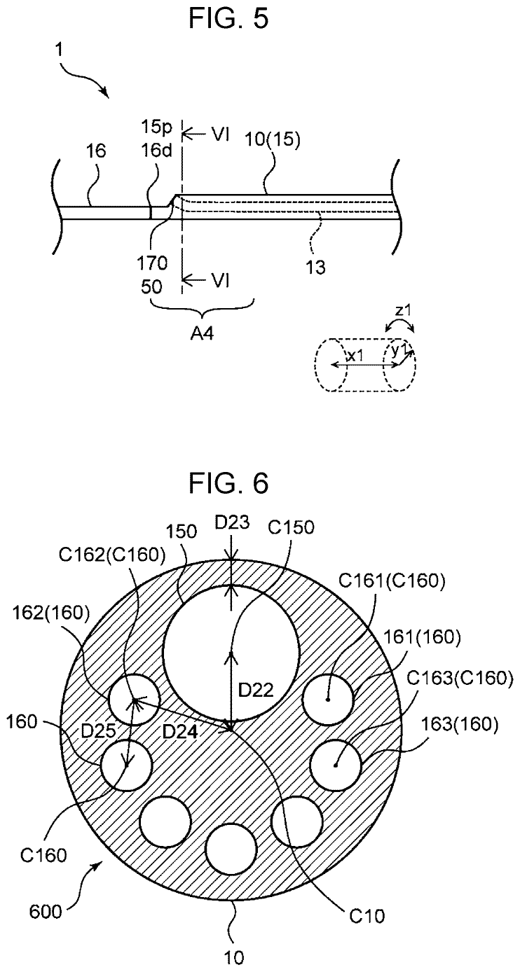

Hereinafter, a balloon catheter according to one or more embodiments of the present invention will be described with reference to FIG. 1 to FIG. 6. FIG. 1 is a side view of a balloon catheter according to one or more embodiments of the present invention, and FIG. 2 is a side view of balloons of the balloon catheter illustrated in FIG. 1. FIG. 3 is a side view of a shaft in a distal region of the balloon catheter illustrated in FIG. 1, and FIG. 4 is a cross-sectional view of the shaft illustrated in FIG. 3 taken along the line IV-IV, the cross-sectional view indicating a cross-section perpendicular to the longitudinal direction of the shaft. FIG. 5 is a side view of the shaft in a proximal region of the balloon catheter illustrated in FIG. 1, and FIG. 6 is a cross-sectional view of the shaft illustrated in FIG. 5 taken along the line VI-VI, the cross-sectional view indicating a cross-section perpendicular to the longitudinal direction of the shaft.

As illustrated in FIG. 1 and FIG. 2, a balloon catheter 1 includes a shaft 10 extending in the longitudinal direction from the proximal side toward the distal side, and a plurality of balloons 20 disposed at a distal portion of the shaft 10.

The shaft 10 has a longitudinal direction x1, a radial direction y1 connecting a centroid of an outer edge of the shaft 10 and a point on the outer edge in a cross-section perpendicular to the longitudinal direction x1, and a circumferential direction z1 along the outer edge of the shaft 10 in the cross-section perpendicular to the longitudinal direction x1. In the present description, in the longitudinal direction x1, a direction on the hand side of a user is referred to as the proximal side, and a side opposite to the proximal side, that is, a direction on the side of a treatment target is referred to as the distal side.

Members and portions other than the shaft 10 also each have a longitudinal direction, a radial direction, and a circumferential direction, which may be the same as or different from the longitudinal direction x1, the radial direction y1, and the circumferential direction z1 of the shaft 10, respectively. In the present description, for ease of understanding, all members and portions are described as having the same longitudinal direction, radial direction, and circumferential direction as the longitudinal direction x1, the radial direction y1, and the circumferential direction z1 of the shaft 10.

The balloons 20 are connected to a distal portion of the shaft 10. The balloons 20 can be inflated by introducing a fluid through the lumen of the shaft 10, and the balloons 20 can be deflated by discharging the fluid. In order to control the inflation and deflation of the balloons 20, a fluid can be introduced or discharged using an indeflator (balloon pressurizer). As the fluid, for example, physiological saline, a mixed solution of a contrast medium and physiological saline, or the like is used. The fluid may be a pressurized fluid that is pressurized by a pump or the like.

Examples of the material constituting the balloons 20 include polyamide resins such as nylon 11 and nylon 12, polyester resins such as polyethylene terephthalate and polybutylene terephthalate, polyurethane resins, and thermoplastic elastomers such as polyether block amide copolymers.

The materials constituting respective ones of the plurality of balloons 20 may be different from each other but may be the same. That is, the plurality of balloons 20 may be made of the same material. When the materials constituting respective ones of the plurality of balloons 20 are the same, it is possible to make the degree of inflation, hardness, and the like of the balloons 20 substantially the same in the circumferential direction z1 of the balloons 20.

In the inflated state of the balloons 20, respective maximum outer diameters of the plurality of balloons 20 may be different from each other but may be the same. The respective maximum outer diameters of the plurality of balloons 20 being the same means that the respective maximum outer diameters of the plurality of balloons 20 are substantially the same, and to be specific, means that the maximum outer diameter of one balloon 20 is 90% or more and 110% or less of each of the maximum outer diameters of all the other balloons 20. When the respective maximum outer diameters of the plurality of balloons 20 are the same in the inflated state of the balloons 20, the timing of inflation can be easily aligned in all the balloons 20, and the inflation of the balloons 20 can be easily controlled. The inflated state of the balloons 20 means a state in which a fluid is introduced into respective lumens of the plurality of balloons 20 and in which all the balloons 20 are inflated.

As illustrated in FIG. 1 and FIG. 2, the plurality of balloons 20 may include a first balloon 21 and a plurality of second balloons 22 disposed on the outer side with respect to the first balloon 21 to be aligned in the circumferential direction of the first balloon 21. That is, the plurality of balloons 20 include the first balloon 21 and the plurality of second balloons 22, and the plurality of second balloons 22 may be disposed along the outer periphery of the first balloon 21.

The number of the first balloons 21 may be plural but may be one. That is, the balloon catheter 1 may include one first balloon 21 and a plurality of the second balloons 22. When the number of the first balloons 21 is one, the first balloon 21 is less likely to move on the inner side with respect to the plurality of second balloons 22 in the inflated state of the balloons 20. As a result, the inflation of the plurality of second balloons 22 is easily suppressed by the first balloon 21, and the hardness of the balloons 20 is increased, so that the inflation force can be easily increased.

The number of the second balloons 22 may be three or more, four or more, or five or more. By setting the lower limit value of the number of the second balloons 22 in the above range, the positions of the first balloon 21 and the second balloons 22 are less likely to be displaced in the inflated state of the balloons 20, and the inflation force of the balloons 20 can be increased. The number of the second balloons 22 may be 20 or less, 12 or less, 10 or less, or 8 or less. By setting the upper limit value of the number of the second balloons 22 in the above-described range, the outer diameter of the part of the balloons 20 is less likely to be excessively large, and the low invasiveness of the balloon catheter 1 can be improved.

In the inflated state of the plurality of balloons 20, the length from a distal end 20d of each balloon 20 to a proximal end 20p of the balloon 20 in the longitudinal direction x1 may be the same or different among the balloons 20. When the plurality of balloons 20 include the first balloon 21 and the plurality of second balloons 22, a length L2 from the distal end 20d of each second balloon 22 to the proximal end 20p of the second balloon 22 in the longitudinal direction x1 may be different but may be the same among the balloons 20. The fact that respective lengths L2 of the second balloons 22 from the distal end 20d to the proximal end 20p in the longitudinal direction x1 are the Same means that the respective lengths L2 of the plurality of second balloons 22 in the longitudinal direction x1 are substantially the same, and to be specific, means that the length L2 of one second balloon 22 in the longitudinal direction x1 is 90% or more and 110% or less of each of the lengths L2 of all the other second balloons 22 in the longitudinal direction x1. When the respective lengths L2 of the plurality of second balloons 22 in the longitudinal direction x1 in the inflated state of the balloons 20 are the same, the timing of inflation of all the second balloons 22 can be easily aligned, and the inflation of the balloons 20 can be easily controlled.

As illustrated in FIG. 2, the balloons 20 may each include a straight tube portion 203, a proximal-side tapered portion 202 located on the proximal side with respect to the straight tube portion 203, a proximal-side sleeve portion 201 located on the proximal side with respect to the proximal-side tapered portion 202, a distal-side tapered portion 204 located on the distal side with respect to the straight tube portion 203, and a distal-side sleeve portion 205 located on the distal side with respect to the distal-side tapered portion 204.

The straight tube portion 203 may have a substantially cylindrical shape having substantially the same diameters in the longitudinal direction x1 but may have different diameters in the longitudinal direction x1. It is preferable that the proximal-side tapered portion 202 and the distal-side tapered portion 204 are each formed in a substantially conical shape or a truncated conical shape by being reduced in diameter with increasing distance from the straight tube portion 203. When the straight tube portion 203 has a maximum diameter, the straight tube portion 203 of each balloon 20 sufficiently comes into contact with a lesion portion when the balloons 20 are inflated in the lesion portion such as a stenosis portion, and thus it is possible to easily perform treatment such as dilation of the lesion portion. In addition, with the diameters of the proximal-side tapered portion 202 and the distal-side tapered portion 204 being reduced, when the balloons 20 are deflated, the outer diameters of a proximal end portion and a distal end portion of each of the balloons 20 can be reduced to reduce a step between the shaft 10 and each of the balloons 20. Therefore, the outer surface of the balloon catheter 1 becomes smooth in the deflated state of the balloons 20, and the balloon catheter 1 can be easily inserted into a body cavity.

In each balloon 20, the proximal-side sleeve portion 201 and the distal-side sleeve portion 205 may be portions that do not inflate while the proximal-side tapered portion 202, the straight tube portion 203, and the distal-side tapered portion 204 inflate when a fluid is introduced into the balloon 20. When the proximal-side sleeve portion 201 and the distal-side sleeve portion 205 do not inflate, at least a part of the proximal-side sleeve portion 201 and at least a part of the distal-side sleeve portion 205 can be easily fixed to the shaft 10.

In a cross-section at the distal end 10d of the shaft 10 and perpendicular to the longitudinal direction x1, the shaft 10 has an inner lumen 150 and a lumen group 600 constituted by a plurality of outer lumens 160 disposed on the outer side with respect to the inner lumen 150 to be aligned in the circumferential direction z1 of the inner lumen 150.

The inner lumen 150 is a lumen that is disposed on the inner side with respect to the lumen group 600 constituted by the plurality of outer lumens 160 in a cross-section at the distal end 10d of the shaft 10 and perpendicular to the longitudinal direction x1. The inner lumen 150 may be not located on the inner side with respect to the lumen group 600 at a location other than the distal end 10d of the shaft 10, such as at a proximal end 10p of the shaft 10 or a central portion of the shaft 10 in the longitudinal direction x1. The lumen located on the inner side with respect to the lumen group 600 at the distal end 10d of the shaft 10 is referred to as the inner lumen 150.

FIG. 1 illustrates the balloon catheter 1 of a so-called rapid exchange type that has a guidewire port 50 in an intermediate portion between the distal side and the proximal side of the shaft 10 and that includes a guidewire tube 40 functioning as a guidewire insertion path from the guidewire port 50 to the distal side of the shaft 10. The balloon catheter 1 may include a distal-side shaft 15 and a proximal-side shaft 16. The distal-side shaft 15 and the proximal-side shaft 16 may be separate members, and a proximal end portion of the distal-side shaft 15 may be connected to a distal end portion of the proximal-side shaft 16 to constitute the shaft 10 extending from the balloons 20 to a proximal end portion of the balloon catheter 1. Alternatively, one shaft 10 may extend from the balloons 20 to the proximal end portion of the balloon catheter 1, and the distal-side shaft 15 and the proximal-side shaft 16 may be further constituted by a plurality of tube members.

The shaft 10 may be made of a resin, a metal, or a combination of a resin and a metal. By using a resin as the constituent material of the shaft 10, flexibility and elasticity can be easily imparted to the shaft 10. In addition, by using a metal as the constituent material of the shaft 10, the deliverability of the balloon catheter 1 can be improved. Examples of the resin constituting the shaft 10 include polyamide-based resin, polyester resin, polyurethane-based resin, polyolefin-based resin, fluorine-based resin, vinyl chloride resin, silicone-based resin, natural rubber, and synthetic rubbers. These may be used alone or in combination of two or more. Examples of a metal constituting the shaft 10 include stainless steels such as SUS304 or SUS316, platinum, nickel, cobalt, chromium, titanium, tungsten, gold, Ni-Ti alloy, Co-Cr alloy, and a combination thereof. When the shaft 10 includes the distal-side shaft 15 and the proximal-side shaft 16 which are separate members, for example, the distal-side shaft 15 can be formed of a resin while the proximal-side shaft 16 is formed of a metal. Further, the shaft 10 may have a laminated structure made of different materials or the same material.

The balloons 20 and the shaft 10 are joined to each other by, for example, adhesion with an adhesive, welding, or crimping with a ring-shaped member attached to a portion where an end portion of each balloon 20 and the shaft 10 overlap each other. In particular, each balloon 20 and the shaft 10 may be joined to each other by welding. When each balloon 20 and the shaft 10 are joined to each other by welding, the joining between each balloon 20 and the shaft 10 is less likely to be released even when the balloons 20 are repeatedly inflated or deflated, and it is thus possible to improve the joining strength.

As illustrated in FIG. 1, a hub 5 may be provided on the proximal side with respect to the shaft 10. In addition, the hub 5 may be provided with a fluid injection portion 6 in communication with a flow path of a fluid to be supplied to the inside of each balloon 20.

The shaft 10 and the hub 5 are joined to each other by, for example, adhesion with an adhesive, welding, or the like. In particular, the shaft 10 and the hub 5 may be joined to each other by adhesion. When the shaft 10 and the hub 5 are joined to each other by adhesion, the joining strength between the shaft 10 and the hub 5 can be increased to improve the durability of the balloon catheter 1 when the material of the shaft 10 is different from the material of the hub 5, for example, when the shaft 10 is made of a highly flexible material and the hub 5 is made of a highly rigid material.

The inner lumen 150 and the outer lumens 160 extend in the longitudinal direction x1. In a cross-section perpendicular to the longitudinal direction x1 of the shaft 10, the cross-sectional area of the inner lumen 150 may be larger than the cross-sectional area of each outer lumen 160. When the cross-sectional area of the inner lumen 150 is larger than the cross-sectional area of each outer lumen 160, appropriate flexibility can be easily imparted to the shaft 10, the shaft 10 can be easily bent along a curved lumen in a living body, and low invasiveness can be improved.

In a cross-section perpendicular to the longitudinal direction x1 of the shaft 10, the cross-sectional area of the inner lumen 150 may be 1.5 times or more, 2.0 times or more, or 2.5 times or more the cross-sectional area of each outer lumen 160. By setting the lower limit value of the ratio between the cross-sectional area of the inner lumen 150 and the cross-sectional area of each outer lumen 160 within the above-described range, appropriate flexibility can be imparted to the shaft 10, and the shaft 10 can be easily bent along a curved lumen in a living body. In addition, in a cross-section perpendicular to the longitudinal direction x1 of the shaft 10, the cross-sectional area of the inner lumen 150 may be 10 times or less, 9 times or less, or 8 times or less the cross-sectional area of each outer lumen 160. By setting the upper limit value of the ratio between the cross-sectional area of the inner lumen 150 and the cross-sectional area of each outer lumen 160 in the above-described range, the outer diameter of the shaft 10 is less likely to increase, and thus it is possible to improve low invasiveness.

In a cross-section perpendicular to the longitudinal direction x1 of the shaft 10, respective cross-sectional areas of the plurality of outer lumens 160 constituting the lumen group 600 may be different from each other but may be the same. The fact that the cross-sectional areas of the plurality of outer lumens 160 constituting the lumen group 600 are the same means that the cross-sectional areas of the plurality of outer lumens 160 constituting the lumen group 600 are substantially the same, and specifically means that the cross-sectional area of one outer lumen 160 is 90% or more and 110% or less of each of the cross-sectional areas of all the other outer lumens 160. When respective cross-sectional areas of the plurality of outer lumens 160 constituting the lumen group 600 are the same as each other, the rigidity of the shaft 10 in the circumferential direction z1 is substantially the same, and the flexibility of the shaft 10 is likely to be substantially the same in the circumferential direction z1.

As illustrated in FIG. 4, in a cross-section at the distal end 10d of the shaft 10 and perpendicular to the longitudinal direction x1, the lumen group 600 includes a first outer lumen 161, a second outer lumen 162 disposed adjacent to the first outer lumen 161 in the circumferential direction z1 of the inner lumen 150, and a third outer lumen 163 disposed adjacent to the first outer lumen 161 and opposite to the second outer lumen 162 in the circumferential direction z1 of the inner lumen 150. That is, the lumen group 600 includes the first outer lumen 161, the second outer lumen 162 disposed on one side in the circumferential direction z1 of the inner lumen 150 with respect to the first outer lumen 161, and the third outer lumen 163 disposed on the other side in the circumferential direction z1 of the inner lumen 150 with respect to the first outer lumen 161. In other words, the second outer lumen 162, the first outer lumen 161, and the third outer lumen 163 are disposed in this order toward one side in the circumferential direction z1 of the inner lumen 150.

As illustrated in FIG. 4, in a cross-section perpendicular to the longitudinal direction x1, the shaft 10 includes a portion in which a distance D20 between a center C161 of the first outer lumen 161 and a center C162 of the second outer lumen 162 is larger than a distance D21 between the center C161 of the first outer lumen 161 and a center C163 of the third outer lumen 163. The center C161 of the first outer lumen 161 in a cross-section perpendicular to the longitudinal direction x1 refers to the centroid of an outer edge of the cross-sectional shape of the first outer lumen 161. Similarly, the center C162 of the second outer lumen 162 in a cross-section perpendicular to the longitudinal direction x1 refers to the centroid of an outer edge of the cross-sectional shape of the second outer lumen 162, and the center C163 of the third outer lumen 163 in a cross-section perpendicular to the longitudinal direction x1 refers to the centroid of an outer edge of the cross-sectional shape of the third outer lumen 163.

In a cross-section perpendicular to the longitudinal direction x1, since the shaft 10 includes a portion in which the distance D20 between the center C161 of the first outer lumen 161 and the center C162 of the second outer lumen 162 is larger than the distance D21 between the center C161 of the first outer lumen 161 and the center C163 of the third outer lumen 163, the distance between the first outer lumen 161 and the second outer lumen 162 in the circumferential direction z1 of the inner lumen 150 can be larger than the distance between the first outer lumen 161 and the third outer lumen 163 in the circumferential direction z1 of the inner lumen 150. Therefore, in the circumferential direction z1 of the inner lumen 150, the wall thickness of the shaft 10 in the portion between the first outer lumen 161 and the second outer lumen 162 is made larger than the wall thickness of the shaft 10 in the portion between the first outer lumen 161 and the third outer lumen 163, and the rigidity of the portion between the first outer lumen 161 and the second outer lumen 162 of the shaft 10 can be increased. Since the shaft 10 has the increased wall-thickness portion with high rigidity in a certain section in the longitudinal direction x1, a force applied from the proximal side of the shaft 10 is easily transmitted to the distal end 10d of the shaft 10, and the balloon catheter 1 can have favorable force transmissibility.

In a cross-section perpendicular to the longitudinal direction x1, the distance D20 between the center C161 of the first outer lumen 161 and the center C162 of the second outer lumen 162 may be 1.5 times or more, 1.7 times or more, or 2.0 times or more the distance D21 between the center C161 of the first outer lumen 161 and the center C163 of the third outer lumen 163. By setting the lower limit value of the ratio between the distance D20 between the center C161 of the first outer lumen 161 and the center C162 of the second outer lumen 162 and the distance D21 between the center C161 of the first outer lumen 161 and the center C163 of the third outer lumen 163 in the above-described range, the wall thickness of the portion of the shaft 10 between the first outer lumen 161 and the second outer lumen 162 is likely to be increased, and the rigidity of the shaft 10 is likely to be increased. In a cross-section perpendicular to the longitudinal direction x1, the distance D20 between the center C161 of the first outer lumen 161 and the center C162 of the second outer lumen 162 may be 10 times or less, 7 times or less, or 5 times or less the distance D21 between the center C161 of the first outer lumen 161 and the center C163 of the third outer lumen 163. By setting the upper limit value of the ratio between the distance D20 between the center C161 of the first outer lumen 161 and the center C162 of the second outer lumen 162 and the distance D21 between the center C161 of the first outer lumen 161 and the center C163 of the third outer lumen 163 within the above range, the outer diameter of the shaft 10 is less likely to be excessively large, and the low-invasiveness of the balloon catheter 1 can be improved.

In a cross-section perpendicular to the longitudinal direction x1, the shaft 10 may include a portion in which the distance D20 between the center C161 of the first outer lumen 161 and the center C162 of the second outer lumen 162 is larger than a longitudinal diameter of each of the outer lumens 160 constituting the lumen group 600. With the portion in which the distance D20 between the center C161 of the first outer lumen 161 and the center C162 of the second outer lumen 162 is larger than the longitudinal diameter of each of the outer lumens 160 constituting the lumen group 600, the distance between the first outer lumen 161 and the second outer lumen 162 in the circumferential direction z1 of the inner lumen 150 can be easily increased. As a result, the wall thickness of the portion of the shaft 10 between the first outer lumen 161 and the second outer lumen 162 is increased, and it is possible to increase the rigidity of the shaft 10 and improve force transmissibility.

In a cross-section perpendicular to the longitudinal direction x1, the distance D20 between the center C161 of the first outer lumen 161 and the center C162 of the second outer lumen 162 may be 1.1 times or more, 1.2 times or more, or 1.3 times or more the longitudinal diameter of each of the outer lumens 160 constituting the lumen group 600. By setting the lower limit value of the ratio between the distance D20 between the center C161 of the first outer lumen 161 and the center C162 of the second outer lumen 162 and the longitudinal diameter of each of the outer lumens 160 constituting the lumen group 600 within the above range, the first outer lumen 161 and the second outer lumen 162 can be easily spaced apart from each other to ensure the wall thickness sufficiently, and the rigidity of the shaft 10 can be increased. In a cross-section perpendicular to the longitudinal direction x1, the distance D20 between the center C161 of the first outer lumen 161 and the center C162 of the second outer lumen 162 may be 5.0 times or less, 4.5 times or less, or 4.0 times or less the longitudinal diameter of each of the outer lumens 160 constituting the lumen group 600. By setting the upper limit value of the ratio between the distance D20 between the center C161 of the first outer lumen 161 and the center C162 of the second outer lumen 162 and the longitudinal diameter 41 each of the outer lumens 160 constituting the lumen group 600 in the above-described range, it is possible to prevent the outer diameter of the shaft 10 from becoming excessively large and to provide the balloon catheter 1 that is minimally invasive.

As illustrated in FIG. 1, FIG. 3, and FIG. 5, the shaft 10 may have a distal region A3, which is a region on the distal side, and a proximal region A4, which is a region located on the proximal side with respect to the distal region A3.

The distal region A3 includes the distal end 10d of the shaft 10 and is preferably a region extending from the distal end 10d of the shaft 10 toward the proximal side by 1/30 or more of the length from the distal end 10d of the shaft 10 to the proximal end 10p of the shaft 10 in the longitudinal direction x1, more preferably a region extending from the distal end 10d of the shaft 10 toward the proximal side by 1/20 or more of the length from the distal end 10d of the shaft 10 to the proximal end 10p of the shaft 10 in the longitudinal direction x1, and still more preferably a region extending from the distal end 10d of the shaft 10 toward the proximal side by 1/10 or more of the length from the distal end 10d of the shaft 10 to the proximal end 10p of the shaft 10 in the longitudinal direction X1.

The proximal region A4 is located on the proximal side with respect to the distal region A3 and is preferably a region of 1/50 or more of the length from the distal end 10d of the shaft 10 to the proximal end 10p of the shaft 10 in the longitudinal direction x1, more preferably a region of 1/30 or more of the length from the distal end 10d of the shaft 10 to the proximal end 10p of the shaft 10 in the longitudinal direction x1, and even more preferably a region of 1/20 or more of the length from the distal end 10d of the shaft 10 to the proximal end 10p of the shaft 10 in the longitudinal direction x1.

When the shaft 10 includes the distal-side shaft 15 and the proximal-side shaft 16, the distal region A3 includes a distal end 15d of the distal-side shaft 15 and is preferably a region extending from the distal end 15d of the distal-side shaft 15 toward the proximal side by 1/30 or more of the length from the distal end 15d of the distal-side shaft 15 to a proximal end 15p of the distal-side shaft 15 in the longitudinal direction x1, more preferably a region extending from the distal end 15d of the distal-side shaft 15 toward the proximal side by 1/20 or more of the length from the distal end 15d of the distal-side shaft 15 to the proximal end 15p of the distal-side shaft 15 in the longitudinal direction x1, and furthermore preferably a region extending from the distal end 15d of the distal-side shaft 15 toward the proximal side by 1/10 or more of the length from the distal end 15d of the distal-side shaft 15 to the proximal end 15p of the distal-side shaft 15 in the longitudinal direction x1. In addition, the proximal region A4 is located on the proximal side with respect to the distal region A3, includes the proximal end 15p of the distal-side shaft 15, and is preferably a region extending from the proximal end 15p of the distal-side shaft 15 toward the distal side by 1/50 or more of the length from the distal end 15d of the distal-side shaft 15 to the proximal end 15p of the distal-side shaft 15 in the longitudinal direction x1, more preferably a region extending from the proximal end 15p of the distal-side shaft 15 toward the distal side by 1/30 or more of the length from the distal end 15d of the distal-side shaft 15 to the proximal end 15p of the distal-side shaft 15 in the longitudinal direction x1, and furthermore preferably a region extending from the proximal end 15p of the distal-side shaft 15 toward the distal side by 1/20 or more of the length from the distal end 15d of the distal-side shaft 15 to the proximal end 15p of the distal-side shaft 15 in the longitudinal direction x1.

As illustrated in FIG. 6, in a cross-section in the proximal region A4 and perpendicular to the longitudinal direction x1, the distance D20 between the center C161 of the first outer lumen 161 and the center C162 of the second outer lumen 162 may be larger than the distance D21 between the center C161 of the first outer lumen 161 and the center C163 of the third outer lumen 163.

In a cross-section in the proximal region A4 and perpendicular to the longitudinal direction x1, since the distance D20 between the center C161 of the first outer lumen 161 and the center C162 of the second outer lumen 162 is larger than the distance D21 between the center C161 of the first outer lumen 161 and the center C163 of the third outer lumen 163, the distance between the first outer lumen 161 and the second outer lumen 162 in the circumferential direction z1 of the inner lumen 150 can be larger than the distance between the first outer lumen 161 and the third outer lumen 163 in the circumferential direction z1 of the inner lumen 150. In a cross-section perpendicular to the longitudinal direction x1 in the proximal region A4 located at a certain distance from the leading end of the shaft 10, the wall thickness of the portion of the shaft 10 between the first outer lumen 161 and the second outer lumen 162 in the circumferential direction z1 of the inner lumen 150 is made larger than the wall thickness of the portion of the shaft 10 between the first outer lumen 161 and the third outer lumen 163, and the rigidity of the portion of the shaft 10 between the first outer lumen 161 and the second outer lumen 162 can be increased. As a result, the shaft 10 has the increased wall-thickness portion with high rigidity in the proximal region A4, and a force applied from the proximal side of the shaft 10 is easily transmitted to the distal end 10d of the shaft 10, and the transmissibility of the balloon catheter 1 having favorable force transmissibility can be improved.

In the longitudinal direction x1, the length of the proximal region A4 may be less than or equal to the length of the distal region A3. When the length of the proximal region A4 is equal to the length of the distal region A3 or shorter than the length of the distal region A3, the lengths of the proximal region A4 having high flexibility and the distal region A3 having appropriate rigidity are balanced in the entire shaft 10, and the shaft 10 having favorable insertability can be obtained. The length of the proximal region A4 may be shorter than the length of the distal region A3 in the longitudinal direction x1. When the length of the proximal region A4 is shorter than the length of the distal region A3, the insertability of the shaft 10 can be further improved.

As illustrated in FIG. 4 and FIG. 6, a distance D22 between a center C10 of the shaft 10 and a center C150 of the inner lumen 150 in a cross-section in the proximal region A4 and perpendicular to the longitudinal direction x1 may be larger than the distance D22 between the center C10 of the shaft 10 and the center C150 of the inner lumen 150 in a cross-section in the distal region A3 and perpendicular to the longitudinal direction x1. In FIG. 4, the position of the center C10 of the shaft 10 in a cross-section in the distal region A3 and perpendicular to the longitudinal direction x1 coincides with the position of the center C150 of the inner lumen 150, and the distance D22 is zero. Since the distance D22 between the center C10 of the shaft 10 and the center C150 of the inner lumen 150 in the proximal region A4 is larger than the distance D22 between the center C10 of the shaft 10 and the center C150 of the inner lumen 150 in the distal region A3, the position of the inner lumen 150 is closer to the outer periphery of the shaft 10 in the proximal region A4 than in the distal region A3. As a result, in a cross-section perpendicular to the longitudinal direction x1 of the shaft 10, the inner lumen 150 is located in a central portion of the shaft 10 in a distal-side portion of the shaft 10 such as the distal region A3, whereas a partition wall that defines the inner lumen 150 and the outer lumens 160 is present in the central portion of the shaft 10 in a proximal-side portion of the shaft 10 such as the proximal region A4. Therefore, in the proximal-side portion of the shaft 10, the rigidity of the central portion of the shaft 10 in a cross-section perpendicular to the longitudinal direction x1 of the shaft 10 is increased, the shaft 10 is less likely to be bent, and a force that is applied from the proximal end 10p of the shaft 10 is easily transmitted to the distal side. In the shaft 10 illustrated in FIG. 6, the inner lumen 150 is not positioned on the inner side with respect to the lumen group 600 constituted by the plurality of outer lumens 160, and there are the outer lumens 160 positioned on the further inner side with respect to the shaft 10 than the inner lumen 150. The inner lumen 150 is a lumen positioned, as illustrated in FIG. 4, on the inner side with respect to the lumen group 600 constituted by the plurality of outer lumens 160 at the distal end 10d of the shaft 10.

The distance D22 between the center C10 of the shaft 10 and the center C150 of the inner lumen 150 in a cross-section perpendicular to the longitudinal direction x1 may increase in the direction from the distal end 10d toward the proximal end 10p. In other words, it is preferable that the distance D22 between the center C10 of the shaft 10 and the center C150 of the inner lumen 150 in a cross-section perpendicular to the longitudinal direction x1 increases from the distal side toward the proximal side of the shaft 10. When the distance D22 between the center C10 of the shaft 10 and the center C150 of the inner lumen 150 increases in the direction from the distal end 10d toward the proximal end 10p, the position of the inner lumen 150 becomes closer to the outer periphery of the shaft 10 toward the proximal side. Therefore, the rigidity of the central portion of the shaft 10 in a cross-section perpendicular to the longitudinal direction x1 of the shaft 10 increases toward the proximal side of the shaft 10, and the shaft 10 is less likely to bend at the proximal side of the shaft 10. As a result, a force applied from the proximal end 10p of the shaft 10 is easily transmitted to the distal end 10d of the shaft 10, and the balloon catheter 1 can have favorable force transmissibility.

The distance D22 between the center C10 of the shaft 10 and the center C150 of the inner lumen 150 in a cross-section in the proximal region A4 and perpendicular to the longitudinal direction x1 may be 1.1 times or more, 1.2 times or more, or 1.3 times or more the distance D22 between the center C10 of the shaft 10 and the center C150 of the inner lumen 150 in a cross-section in the distal region A3 and perpendicular to the longitudinal direction x1. By setting the lower limit value of the ratio of the distance D22 between the center C10 of the shaft 10 and the center C150 of the inner lumen 150 in the proximal region A4 to the distance D22 between the center C10 of the shaft 10 and the center C150 of the inner lumen 150 in the distal region A3 to the above-described range, the inner lumen 150 can be sufficiently closer to the outer periphery of the shaft 10 in the proximal region A4 than in the distal region A3, and the rigidity at the proximal side of the shaft 10 is easily increased. The distance D22 between the center C10 of the shaft 10 and the center C150 of the inner lumen 150 in a cross-section in the proximal region A4 and perpendicular to the longitudinal direction x1 may be 15 times or less, 10 times or less, or 5 times or less the distance D22 between the center C10 of the shaft 10 and the center C150 of the inner lumen 150 in a cross-section in the distal region A3 and perpendicular to the longitudinal direction x1. By setting the upper limit value of the ratio between the distance D22 between the center C10 of the shaft 10 and the center C150 of the inner lumen 150 in the proximal region A4 and the distance D22 between the center C10 of the shaft 10 and the center C150 of the inner lumen 150 in the distal region A3 in the above-described range, the distance between the outer edge of the inner lumen 150 and the outer surface of the shaft 10 can be easily ensured, and the partition wall between the inner lumen 150 and the outer surface of the shaft 10 can be prevented from being damaged by an article, such as a guidewire, inserted into the inner lumen 150.

As illustrated in FIG. 5, the shaft 10 may have, in the proximal region A4 and in a region between the outer lumens 160 adjacent to each other in the circumferential direction z1 of the inner lumen 150, an opening 170 through which the inner lumen 150 is in communication with a region outside the shaft 10. In this case, as illustrated in FIG. 6, a region where the inner lumen 150 approaches the outer surface side of the shaft 10 is formed in the vicinity of the opening 170. Since the shaft 10 has, in the proximal region A4, the opening 170 in the region between the adjacent outer lumens 160, the balloon catheter 1 becomes a so-called rapid exchange type, and an article such as a guidewire, a fluid, or the like can be easily introduced into the inner lumen 150 through the opening 170. As a result, procedure time in which the balloon catheter 1 is used can be shortened, and the low invasiveness can be improved.

The opening 170 may be located in a region between the first outer lumen 161 and the second outer lumen 162 in the circumferential direction z1 of the inner lumen 150. Since the opening 170 is located in the region between the first outer lumen 161 and the second outer lumen 162, the opening 170 is located between the first outer lumen 161 and the second outer lumen 162 that are spaced apart from each other in the circumferential direction z1 of the inner lumen 150. Therefore, when an article such as a guidewire is inserted into the opening 170, the article inserted into the opening 170 is less likely to come into contact with the partition wall forming the inner lumen 150, and the shaft 10 can be less likely to be damaged.

The opening 170 may be the guidewire port 50 for inserting a guidewire into the inner lumen 150. That is, the balloon catheter 1 of one or more embodiments of the present invention may be a so-called rapid-exchange-type balloon catheter 1.

As illustrated in FIG. 6, in a cross-section in the proximal region A4 and perpendicular to the longitudinal direction x1, the distance D22 between the center C10 of the shaft 10 and the center C150 of the inner lumen 150 may be larger than a shortest distance D23 between the outer edge of the inner lumen 150 and the outer edge of the shaft 10. The shortest distance D23 between the outer edge of the inner lumen 150 and the outer edge of the shaft 10 in a cross-section perpendicular to the longitudinal direction x1 refers to a length of the shortest one of straight lines each connecting a point on the outer edge of the inner lumen 150 to a point on the outer edge of the shaft 10. Since the distance D22 between the center C10 of the shaft 10 and the center C150 of the inner lumen 150 is larger than the shortest distance D23 between the outer edge of the inner lumen 150 and the outer edge of the shaft 10, the inner lumen 150 is likely to be close to the outer periphery of the shaft 10 in the proximal region A4. Therefore, the rigidity of a center portion of the shaft 10 is easily increased, and a force applied from the proximal side of the shaft 10 is easily transmitted to the distal side.

In a cross-section in the proximal region A4 and perpendicular to the longitudinal direction x1, the distance D22 between the center C10 of the shaft 10 and the center C150 of the inner lumen 150 may be 1.1 times or more, 1.2 times or more, or 1.3 times or more the shortest distance D23 between the outer edge of the inner lumen 150 and the outer edge of the shaft 10. By setting the lower limit value of the ratio between the distance D22 between the center C10 of the shaft 10 and the center C150 of the inner lumen 150 in the proximal region A4 and the shortest distance D23 between the outer edge of the inner lumen 150 and the outer edge of the shaft 10 to the above-described range, the inner lumen 150 easily approaches the outer periphery of the shaft 10 in the proximal region A4, and the rigidity of a center portion of the shaft 10 easily increases. In a cross-section in the proximal region A4 and perpendicular to the longitudinal direction x1, the distance D22 between the center C10 of the shaft 10 and the center C150 of the inner lumen 150 may be 10 times or less, 7 times or less, or 5 times or less the shortest distance D23 between the outer edge of the inner lumen 150 and the outer edge of the shaft 10. By setting the upper limit value of the ratio between the distance D22 between the center C10 of the shaft 10 and the center C150 of the inner lumen 150 in the proximal region A4 and the shortest distance D23 between the outer edge of the inner lumen 150 and the outer edge of the shaft 10 within the above-described range, the partition wall between the inner lumen 150 and the outer edge of the shaft 10 can be easily ensured in the proximal region A4, and the shaft 10 can be less likely to be damaged when an article such as a guidewire is inserted into the inner lumen 150.

As illustrated in FIG. 6, in a cross-section in the proximal region A4 and perpendicular to the longitudinal direction x1, a shortest distance D24 between a center C160 of each of the outer lumens 160 constituting the lumen group 600 and the center C10 of the shaft 10 may be larger than a center-to-center distance D25 between the outer lumens 160 adjacent to each other in the circumferential direction z1 of the inner lumen 150. The shortest distance D24 between the center C160 of each outer lumen 160 and the center C10 of the shaft 10 in a cross-section perpendicular to the longitudinal direction x1 refers to a length of the shortest one of straight lines each connecting the center C160 of each outer lumen 160 to the center C10 of the shaft 10 in the outer lumens 160. Since the shortest distance D24 between the center C160 of each outer lumen 160 and the center C10 of the shaft 10 in the proximal region A4 is larger than the center-to-center distance D25 between the outer lumens 160 adjacent to each other, the outer lumens 160 can easily approach the outer periphery of the shaft 10, and the rigidity of a center portion of the shaft 10 can be easily increased. As a result, the rigidity of the shaft 10 in the proximal region A4 is increased, and a force that is applied to the shaft 10 from the proximal side toward the distal side can be easily transmitted to the distal end 10d of the shaft 10.

In a cross-section in the proximal region A4 and perpendicular to the longitudinal direction x1, the shortest distance D24 between the center C160 of each of the outer lumens 160 constituting the lumen group 600 and the center C10 of the shaft 10 may be 1.1 times or more, 1.2 times or more, or 1.3 times or more the center-to-center distance D25 between the outer lumens 160 adjacent to each other in the circumferential direction z1 of the inner lumen 150. By setting the lower limit value of the ratio between the shortest distance D24 between the center C160 of each outer lumen 160 and the center C10 of the shaft 10 and the center-to-center distance D25 between the outer lumens 160 adjacent to each other to the above-described range, the outer lumens 160 can be easily brought close to the outer periphery of the shaft 10 in the proximal region A4, and the rigidity of a center portion of the shaft 10 can be easily increased. In addition, in a cross-section perpendicular to the longitudinal direction x1 in the proximal region A4, the shortest distance D24 between the center C160 of each of the outer lumens 160 constituting the lumen group 600 and the center C10 of the shaft 10 may be 10 times or less, 9 times or less, or 8 times or less the center-to-center distance D25 between the outer lumens 160 adjacent to each other in the circumferential direction z1 of the inner lumen 150. By setting the upper limit value of the ratio between the shortest distance D24 between the center C160 of each outer lumen 160 and the center C10 of the shaft 10 and the center-to-center distance D25 between the adjacent outer lumens 160 in the above-described range, the outer diameter of the shaft 10 is less likely to become excessively large, and the balloon catheter 1 that is minimally invasive can be obtained.

As illustrated in FIG. 6, in a cross-section in the proximal region A4 and perpendicular to the longitudinal direction x1, the shortest distance D24 between the center C160 of each of the outer lumens 160 constituting the lumen group 600 and the center C10 of the shaft 10 may be larger than a shortest distance D26 between the outer edge of each outer lumen 160 and the outer edge of the shaft 10. The shortest distance D26 between the outer edge of each outer lumen 160 and the outer edge of the shaft 10 in a cross-section perpendicular to the longitudinal direction x1 refers to a length of the shortest one of straight lines each connecting a point on the outer edge of the outer lumen 160 and a point on the outer edge of the shaft 10 in the outer lumens 160. When the shortest distance D24 between the center C160 of each outer lumen 160 and the center C10 of the shaft 10 is larger than the shortest distance D26 between the outer edge of each outer lumen 160 and the outer edge of the shaft 10, the outer lumens 160 are easily separated from the center C10 of the shaft 10 in the proximal region A4. As a result, the rigidity of a center portion of the shaft 10 in the proximal region A4 is easily increased, and a force that is applied from the proximal side of the shaft 10 can be easily transmitted to the distal side.

In a cross-section in the proximal region A4 and perpendicular to the longitudinal direction x1, the shortest distance D24 between the center C160 of each of the outer lumens 160 constituting the lumen group 600 and the center C10 of the shaft 10 may be 1.5 times or more, 2.0 times or more, or 2.5 times or more the shortest distance D26 between the outer edge of each outer lumen 160 and the outer edge of the shaft 10. By setting the lower limit value of the ratio between the shortest distance D24 between the center C160 of each outer lumen 160 and the center C10 of the shaft 10 and the shortest distance D26 between the outer edge of each outer lumen 160 and the outer edge of the shaft 10 within the above range, the center C160 of each outer lumen 160 and the center C10 of the shaft 10 can be easily separated from each other by a large distance, and the rigidity of a center portion of the shaft 10 can be sufficiently increased. In addition, in a cross-section in the proximal region A4 and perpendicular to the longitudinal direction x1, the shortest distance D24 between the center C160 of each of the outer lumens 160 constituting the lumen group 600 and the center C10 of the shaft 10 may be 20 times or less, 18 times or less, or 15 times or less the shortest distance D26 between the outer edge of each outer lumen 160 and the outer edge of the shaft 10. By setting the upper limit value of the ratio between the shortest distance D24 between the center C160 of each outer lumen 160 and the center C10 of the shaft 10 and the shortest distance D26 between the outer edge of each outer lumen 160 and the outer edge of the shaft 10 within the above range, the thickness of the partition wall between each outer lumen 160 and the outer periphery of the shaft 10 can be easily ensured, and the partition wall of each outer lumen 160 is less likely to be damaged when, for example, the shaft 10 is bent.

As illustrated in FIG. 3 and FIG. 5, the inner lumen 150 may be a guidewire lumen 13 through which a guidewire is to be inserted, the outer lumens 160 constituting the lumen group 600 are inflation lumens through which a fluid to be supplied to the lumens of the balloons 20 passes, and the outer lumens 160 each communicate with a corresponding one of the lumens of the plurality of balloons 20. When the inner lumen 150 is the guidewire lumen 13 and the outer lumens 160 are the inflation lumens, it is possible to easily insert a guidewire into the inner lumen 150 and to make the shaft 10 less likely to be damaged when a guidewire is inserted into the inner lumen 150.

As illustrated in FIG. 1 and FIG. 2, a guidewire tube 40 having a lumen communicating with the guidewire lumen 13 may be further included, and the guidewire tube 40 may be disposed in the lumen of one of the balloons 20. When the balloon catheter 1 includes the guidewire tube 40 having the lumen communicating with the guidewire lumen 13, a guidewire can be easily inserted into the balloon catheter 1, and the balloon catheter 1 can be conveyed along the guidewire into a body. Further, by inserting a guidewire into the guidewire tube 40, it is possible to prevent the guidewire from damaging the balloons 20 and the like.

Examples of the material constituting the guidewire tube 40 include synthetic resins and the like including polyolefin-based resin such as polyethylene and polypropylene; polyamide-based resin such as nylon; polyester-based resin such as PET; aromatic polyether ketone-based resin such as PEEK; polyether polyamide-based resin; polyurethane-based resin; polyimide-based resin; fluorine-based resin such as PTFE, PFA, and ETFE; and polyvinyl chloride-based resin. In particular, the material constituting the guidewire tube 40 may be the polyimide-based resin. When the material constituting the guidewire tube 40 is the polyimide-based resin, the slidability of the guidewire tube 40 is improved. Therefore, it is easy to insert a guidewire into the lumen of the guidewire tube 40 and to advance the balloon catheter 1 into a body along the guidewire. Further, the guidewire tube 40 may have a multi-layer structure including a braid layer such as a metal braid. When the guidewire tube 40 has a multilayer structure, the strength, the slidability with respect to a guidewire, and the kink resistance of the guidewire tube 40 can be enhanced.

As illustrated in FIG. 1, a proximal end portion of the guidewire tube 40 may be connected to a distal end portion of the shaft 10. When the shaft 10 is configured to include the distal-side shaft 15 and the proximal-side shaft 16, a proximal end portion of the guidewire tube 40 may be connected to a distal end portion of the distal-side shaft 15. When the proximal end portion of the guidewire tube 40 is connected to the distal end portion of the shaft 10, the outer diameter of the balloon catheter 1 is less likely to be large, and the low invasiveness can be improved.

The distal end portion of the balloon catheter 1 may be provided with an end tip member 60. The end tip member 60 may be provided, as a separate member from the guidewire tube 40, at a distal end portion of the balloon catheter 1 by being connected to distal end portions of the balloons 20, or the guidewire tube 40 extending to the distal side further than the distal ends 20d of the balloons 20 may function as the end tip member 60.

As illustrated in FIG. 1 and FIG. 2, radiopaque markers 70 may be disposed on the guidewire tube 40 inside the balloon 20 to be located at portions where the balloons 20 are located in the longitudinal direction x1 so that the positions of the balloons 20 can be confirmed by x-ray fluoroscopy.

Examples of the positions on the guidewire tube 40 at which the radiopaque markers 70 are disposed include the position of a midpoint of a length from the distal ends 20d of the balloons 20 to the proximal ends 20p of the balloons 20, and the positions of a proximal end 203p and a distal end 203d of the straight tube portion 203 of each of the balloons 20. In particular, the positions on the guidewire tube 40 at which the radiopaque markers 70 are disposed may be the positions of the proximal end 203p and the distal end 203d of the straight tube portion 203 of each of the balloons 20. When the radiopaque markers 70 are disposed, on the guidewire tube 40, at positions at the proximal end 203p and the distal end 203d of the straight tube portion 203 of each balloon 20, the positions of the balloons 20 can be easily confirmed. As a result, it is possible to provide the balloon catheter 1 capable of easily applying pressure to a target site.

In the shaft 10, an outer wall of at least one of the distal-side shaft 15 and the proximal-side shaft 16 may be coated, and it is more preferable that outer walls of both the distal-side shaft 15 and the proximal-side shaft 16 are coated.

The coating applied to the shaft 10 can be a hydrophilic coating or a hydrophobic coating depending on the purpose of coating, and the coating can be applied by immersing the shaft 10 in a hydrophilic coating agent or a hydrophobic coating agent, applying a hydrophilic coating agent or a hydrophobic coating agent to an outer wall of the shaft 10, or coating the outer wall of the shaft 10 with a hydrophilic coating agent or a hydrophobic coating agent. The coating agent may contain chemicals, additives, and the like.

Examples of the hydrophilic coating agent include hydrophilic coating agents made of hydrophilic polymers such as polyvinyl alcohol, polyethylene glycol, polyacrylamide, polyvinylpyrrolidone, and methyl vinyl ether-maleic anhydride copolymer, or any combination thereof.

Examples of the hydrophobic coating agent include polytetrafluoroethylene (PTFE), fluorinated ethylene propylene (FEP), perfluoroalkoxyalkane (PFA), silicone oil, hydrophobic urethane resin, carbon coating, diamond coating, diamond-like carbon (DLC) coating, ceramic coating, and a substance terminated with an alkyl group or a perfluoroalkyl group and having low surface free energy.

The balloon catheter 1 of one or more embodiments of the present invention may be used for dilation of an aortic valve, deformation of a bioprosthetic valve placed in a heart, or destruction of the bioprosthetic valve. Specifically, it is preferable that the balloon catheter 1 of one or more embodiments of the present invention is used for the purpose of dilating an aortic valve hardened by calcification or the like, or deforming or destroying an artificial valve annulus the like of a bioprosthetic valve to replace the bioprosthetic valve that is placed in a heart and that is deteriorated. The balloon catheter 1 of one or more embodiments of the present invention can have a structure including a plurality of the balloons 20 because the partition wall between the inner lumen 150 and each of the outer lumens 160 of the shaft 10 is less likely to be damaged and a fluid or an article fed into the inner lumen 150 is less likely to enter the outer lumens 160, and can be suitably used because dilation of a hardened aortic valve or deformation or destruction of a bioprosthetic valve, which cannot be sufficiently performed by a conventional balloon catheter, can be easily performed.

The present application claims the benefit of priority based on Japanese Patent Application No. 2023-102819 filed on Jun. 22, 2023. The entire disclosure of Japanese Patent Application No. 2023-102819 filed on Jun. 22, 2023 is incorporated herein by reference.

Although the disclosure has been described with respect to only a limited number of embodiments, those skilled in the art, having benefit of this disclosure, will appreciate that various other embodiments may be devised without departing from the scope of the present disclosure. Accordingly, the scope of the invention should be limited only by the attached claims.

### REFERENCE SIGNS LIST

1 balloon catheter
5 hub
6 fluid injection portion
10 shaft
10d distal end of shaft
10p proximal end of shaft
13 guidewire lumen
15 distal-side shaft
16 proximal-side shaft
20 balloon
20d distal end of balloon
20p proximal end of balloon
201 proximal-side sleeve portion
201d distal end of proximal-side sleeve portion
201p proximal end of proximal-side sleeve portion
202 proximal-side tapered portion
202d distal end of proximal-side tapered portion
202p proximal end of proximal-side tapered portion
203 straight tube portion
203d distal end of straight tube portion
203p proximal end of straight tube portion
204 distal-side tapered portion
204d distal end of distal-side tapered portion
204p proximal end of distal-side tapered portion
205 distal-side sleeve portion
205d distal end of distal-side sleeve portion
205p proximal end of distal-side sleeve portion
21 first balloon
22 second balloon
40 guidewire tube
50 guidewire port
60 end tip member
70 radiopaque marker
150 inner lumen
160 outer lumen
161 first outer lumen
162 second outer lumen
163 third outer lumen
170 opening
600 lumen group
C10 center of shaft
C150 center of inner lumen
C160 center of outer lumen
C161 center of first outer lumen
C162 center of second outer lumen
C163 center of third outer lumen
D20 distance between center of first outer lumen and center of second outer lumen
D21 distance between center of first outer lumen and center of third outer lumen
D22 distance between center of shaft and center of inner lumen
D23 shortest distance between outer edge of inner lumen and outer edge of shaft
D24 shortest distance between center of outer lumen and center of shaft
D25 center-to-center distance between adjacent outer lumens
D26 shortest distance between outer edge of outer lumen and outer edge of shaft
A3 distal region
A4 proximal region
L2 length from distal end of balloon to proximal end of balloon

1. A balloon catheter comprising:
a shaft extending from a proximal side toward a distal side in a longitudinal direction; and
a plurality of balloons disposed at a distal portion of the shaft,
wherein, in a cross-section at a distal end of the shaft and perpendicular to the longitudinal direction, the shaft has an inner lumen and a lumen group constituted by a plurality of outer lumens disposed on an outer side with respect to the inner lumen and aligned in a circumferential direction of the inner lumen,
wherein, in a cross-section at a distal end of the shaft and perpendicular to the longitudinal direction, the lumen group includes a first outer lumen, a second outer lumen disposed adjacent to the first outer lumen in the circumferential direction of the inner lumen, and a third outer lumen disposed adjacent to the first outer lumen and on a side opposite to the second outer lumen in the circumferential direction of the inner lumen, and
wherein, in a cross-section perpendicular to the longitudinal direction, the shaft includes a portion in which a distance between a center of the first outer lumen and a center of the second outer lumen is larger than a distance between a center of the first outer lumen and a center of the third outer lumen.

2. The balloon catheter according to claim 1, wherein, in a cross-section perpendicular to the longitudinal direction, the shaft includes a portion in which a distance between a center of the first outer lumen and a Center of the second outer lumen is larger than a longitudinal diameter of each of the outer lumens constituting the lumen group.

3. The balloon catheter according to claim 1,
wherein the shaft has a distal region, which is a region on a distal side, and a proximal region, which is a region located on a proximal side with respect to the distal region, and
wherein, in a cross-section in the proximal region and perpendicular to the longitudinal direction, a distance between a center of the first outer lumen and a center of the second outer lumen is larger than a distance between a center of the first outer lumen and a center of the third outer lumen.

4. The balloon catheter according to claim 3, wherein a length of the proximal region is equal to or less than a length of the distal region in the longitudinal direction.

5. The balloon catheter according to claim 1,
wherein the shaft has a distal region, which is a region on a distal side, and a proximal region, which is a region located on a proximal side with respect to the distal region, and
wherein a distance between a center of the shaft and a center of the inner lumen in a cross-section in the proximal region and perpendicular to the longitudinal direction is larger than a distance between a center of the shaft and a center of the inner lumen in a cross-section in the distal region and perpendicular to the longitudinal direction.

6. The balloon catheter according to claim 5, wherein the shaft has, in the proximal region and in a region between the outer lumens adjacent to each other in the circumferential direction of the inner lumen, an opening through which the inner lumen is in communication with a region outside the shaft.

7. The balloon catheter according to claim 6, wherein the opening is located in a region between the first outer lumen and the second outer lumen in the circumferential direction of the inner lumen.

8. The balloon catheter according to claim 5, wherein, in a cross-section in the proximal region and perpendicular to the longitudinal direction, a distance between a center of the shaft and a center of the inner lumen is larger than a shortest distance between an outer edge of the inner lumen and an outer edge of the shaft.

9. The balloon catheter according to claim 5, wherein, in a cross-section in the proximal region and perpendicular to the longitudinal direction, a shortest distance between a center of each of the outer lumens constituting the lumen group and a center of the shaft is larger than a center-to-center distance between the outer lumens adjacent to each other in the circumferential direction of the inner lumen.

10. The balloon catheter according to claim 5, wherein, in a cross-section in the proximal region and perpendicular to the longitudinal direction, a shortest distance between a center of each of the outer lumens constituting the lumen group and a center of the shaft is larger than a shortest distance between an outer edge of each of the outer lumens and an outer edge of the shaft.

11. The balloon catheter according to claim 1,
wherein the inner lumen is a guidewire lumen through which a guidewire is configured to be inserted, and
wherein the outer lumens constituting the lumen group are inflation lumens through which a fluid to be supplied to lumens of the balloons is configured to pass, the inflation lumens each being in communication with a corresponding one of lumens of the plurality of balloons.

12. The balloon catheter according to claim 11, further comprising:
a guidewire tube having a lumen in communication with the guidewire lumen,
wherein the guidewire tube is disposed in a lumen of one of the balloons.

13. A method comprising dilating an aortic valve, deforming a bioprosthetic valve placed in a heart, or destroying the bioprosthetic valve, each being performed using the balloon catheter according to claim 1.
